(19) United States
(12) Patent Application Publication
(10) Pub. No.: US 2016/0130614 A1
(43) Pub. Date: May 12, 2016

(54) METHOD OF ENHANCED BIOPRODUCTION

(71) Applicant: Cargill, Incorporated, Wayzata, MN (US)

(72) Inventors: Hendrikus Johannus Meerman, Louisville, CO (US); Hans Liao, Superior, CO (US)

(21) Appl. No.: 14/949,572

(22) Filed: Nov. 23, 2015

Related U.S. Application Data
(62) Division of application No. 14/212,114, filed on Mar. 14, 2014, now abandoned.
(60) Provisional application No. 61/800,821, filed on Mar. 15, 2013.

(51) Int. Cl. C12P 7/42

(52) U.S. Cl. CPC C12P 7/42

(57) ABSTRACT

Bio-based renewable 3-hydroxypropionic acid (3-HP) may be produced through fermentation processes utilizing genetically modified microorganisms such as, for example, genetically modified E. coli strains. The practice of the invention may include cultivating or culturing (meant to be synonymous) cells or genetically modified microorganisms, including in large-scale fermentations.

### CROSS REFERENCE

This application is a divisional of U.S. Ser. No. 14/212,114, filed Mar. 14, 2014, which claims the benefit of U.S. Provisional Application No. 61/800,821, filed Mar. 15, 2013, all of which are incorporated herein by reference in their entirety.

### BACKGROUND OF THE INVENTION

Efforts are increasing to develop microbial fermentation alternatives to production of industrial chemicals and fuels that currently are largely derived from petroleum. These efforts include the use of metabolic engineering approaches to improve performance of such fermentation alternatives.

As fermentation models are refined toward reaching economic viability on an at-cost replacement basis for petro-based chemicals, microbial performance, including production rate and efficiency, remains as a target for improvement. The performance based on any one improvement may require coordination with other modifications.

Notwithstanding advances in the field, there remains a need to further improve microbial performance particularly with regard to improving production of 3-hydroxypropionic acid, which can be converted to many useful monomers (including acrylic acid), industrial chemicals, and consumer products.

### SUMMARY

This disclosure provides for a method of producing 3-hydroxypropionic acid (3-HP) in a fermentation processes comprising culturing an organism and a carbon source in the presence of a non-potassium containing carbonate titrant to control the pH of the fermentation.

In some embodiments, a method of producing 3-hydroxypropionic acid (3-HP) comprises introducing a recombinant microorganism into an industrial bio-production system where the microorganism converts a carbon source into 3-HP wherein the bio-production system includes the introduction of said recombinant microorganism into a bioreactor vessel with the carbon source and bio-production media suitable for growing the recombinant microorganism, and maintaining the bio-production system within a suitable temperature range for a suitable time to obtain a desired conversion of a portion of the substrate molecules to the chemical product while using a non-potassium containing carbonate titrant to control the pH within the bioreactor vessel.

In some embodiments carbonate has a pH of greater than or equal to about 9.5 or 10.

In some embodiments carbonate has a water solubility of greater than or equal to about 1 mole/L at 30° C. In some embodiments, carbonate contains sodium, magnesium or calcium.

In some embodiments, carbonate is selected from the group consisting of sodium carbonate, sodium bicarbonate, and sodium sesquicarbonate, magnesium carbonate, magnesium bicarbonate, calcium carbonate, and calcium bicarbonate. In some embodiments, carbonate is used either alone or in combination with another base.

In some embodiments, the methods of this disclosure produces about 25% to about 50% more 3-HP compared to the same process utilizing ammonia hydroxide as the titrant.

In some embodiments, this disclosure provides for an organism that is a bacteria or yeast. In some embodiments the organism is a E. coli, Cupriavidus necator, or Saccharomyces.

In some embodiments the organism is genetically modified wherein the genetic modification includes introduction of nucleic acid sequences coding for polynucleotides encoding a gene that down regulates one or more of the enzymes used in the TCA cycle. In some embodiments the TCA cycle is selected from the group consisting of citrate synthase (gltA), citrate hydro-lyase (acnA, acnB), isocitrate lyase (aceA), isocitrate dehydrogenase (icd), 2-oxoglutarate dehydrogenase (lpd), succinyl-CoA synthetase (sucD, sucC), succinate dehydrogenase (sdhA, sdhB, sdhC, sdhD), fumarase (fumA, fumB, fumC), malate synthase (aceB), and malate dehydrogenase (mdh).

In some embodiments, the organism is genetically modified wherein the genetic modification includes introduction of nucleic acid sequences coding for polynucleotides encoding a gene that down regulates an enzyme that leads to the product of carbon dioxide, which may be selected from the group consisting of citrate synthase (gltA), citrate hydro-lyase (acnA, acnB), isocitrate lyase (aceA), isocitrate dehydrogenase (icd), and 2-oxoglutarate dehydrogenase (lpd).

In some embodiments, the genetic modification of an organism includes introduction of nucleic acid sequences coding for polynucleotides encoding a gene that acts as a carbon dioxide importer.

In some embodiments, the carbon dioxide importer increases the organism's intracellular carbon dioxide. In some embodiments, the organism is genetically modified wherein the genetic modification includes introduction of nucleic acid sequences coding for polynucleotides encoding one or more heterologous genes selected from the group consisting of bicA from Synechococcus species, ychM gene from E. coli, and yidE gene from E. coli.

In some embodiments, the method is practiced in a large-scale fermentation vessel, wherein the vessel may be greater than 250 L, greater than 1,000 L, 10,000 L, 50,000 L, 100,000 L or 200,000 L.

In some embodiments, 3-hydroxypropionic acid (3-HP) in a fermentation processes comprising culturing an organism and a carbon source in the presence of a titrant to control the pH of the fermentation, wherein said titrant enhances the redox potential of NADH or NADPH.

In some embodiments, the dissolved oxygen concentration within the bioreactor vessel is maintained within an appropriate range.

In some embodiments, the bioproduction is performed under aerobic, microaerobic, or anaerobic conditions, with or without agitation.

In some embodiments, 3-hydroxypropionic acid (3-HP) comprising introducing a recombinant E. coli microorganism into an industrial bio-production system where the microorganism converts a carbon source into 3-HP wherein the bio-production system includes the introduction of said recombinant microorganism into a bioreactor vessel with the carbon source and bio-production media suitable for growing the recombinant microorganism, and maintaining the bio-production system within a suitable temperature range for a suitable time to obtain a desired conversion of a portion of the substrate molecules to the chemical product while using a non-potassium containing carbonate titrant to control the pH within the bioreactor vessel, and wherein said carbonate has a pH of greater than or equal to about 9.5, has a water solubility of greater than or equal to about 1 mole/L at 30° C., and contains sodium, magnesium or calcium.

In some embodiments, the carbonate is selected from the group consisting of sodium carbonate, sodium bicarbonate, and sodium sesquicarbonate, magnesium carbonate, magnesium bicarbonate, calcium carbonate, and calcium bicarbonate.

In some embodiments, the carbonate is used either alone or in combination with another base.

In some embodiments, the method produces about 25% to about 50% more 3-HP compared to the same process utilizing ammonia hydroxide as the titrant.

In some embodiments, the organism is genetically modified wherein the genetic modification includes introduction of nucleic acid sequences coding for polynucleotides encoding: (1) a gene that down regulates one or more of the enzymes used in the TCA cycle selected from the group consisting of citrate synthase (gltA), citrate hydro-lyase (acnA, acnB), isocitrate lyase (aceA), isocitrate dehydrogenase (icd), 2-oxoglutarate dehydrogenase (lpd), succinyl-CoA synthetase (sucD, sucC), succinate dehydrogenase (sdhA, sdhB, sdhC, sdhD), fumarase (fumA, fumB, fumC), malate synthase (aceB), and malate dehydrogenase (mdh); and (2) one or more heterologous genes selected from the group consisting of bicA from Synechococcus species, ychM gene from E. coli, and yidE gene from E. coli.

In some embodiments, the methods of the disclosure are practiced in a large-scale fermentation vessel.

In some embodiments, the dissolved oxygen concentration within the bioreactor vessel is maintained within an appropriate range.

In some embodiments, the bioproduction is performed under aerobic, microaerobic, or anaerobic conditions, with or without agitation.

In some embodiments, bioproduction is performed under aerobic, microaerobic, or anaerobic conditions, with or without agitation.

In some embodiments, the bioproduction system includes a growth phase and a production phase, wherein the organism replicates during the growth phase, and the organism produces 3-HP during the production phase. In some embodiments, the growth phase is conducted at a temperature of about 25 to about 35, about 28 to about 32, or about 30° C.

In some embodiments, the production phase is conducted at a temperature of about 35 to about 45, about 35 to about 40, or about 36 to about 38° C. In some embodiments, the production phase temperature is higher than the growth phase temperature. In some embodiments, the increase in temperature between the production phase and the growth phase occurs over a period of about 1 to about 5 hours, about 1 to about 3 hours, about 2 hours, or about 1 hour.

### BRIEF DESCRIPTION OF THE DRAWINGS

FIG. 1 illustrates biochemical conversion of Acetyl-CoA to malonyl-CoA.

FIG. 2 illustrates parts of the glucose metabolism pathway.

FIG. 3 shows the results of a study conducted to determine the effect of using sodium carbonate versus ammonium hydroxide as the base titrant during the production phase of the fermentation to produce 3-HP.

FIG. 4 shows the effect of using various base titrants on 3HP production.

FIG. 5 shows the effect of using various base titrants on normalized 3HP production.

FIG. 6 shows the effect of using excess ammonium in the base titrant on 3-HP production. The Y-axis shows overall yield at ppp.

FIG. 7 shows the effect of using excess ammonium in the base titrant on 3-HP production. The Y-axis shows overall specific productivity.

FIG. 8 shows the effect of using excess ammonium in the base titrant on 3-HP production. The Y-axis shows average specific rate at ppp.

FIG. 9 shows the results of a study to determine whether the addition of sodium carbonate to a 3-HP shake flask fermentation would result in higher 3-HP titers and prolonged 3-HP production.

### DETAILED DESCRIPTION

Bio-based renewable 3-hydroxypropionic acid (3-HP) may be produced through fermentation processes utilizing genetically modified microorganisms such as, for example, genetically modified E. coli strains. The practice of the invention may include cultivating or culturing (meant to be synonymous) cells, including in large-scale fermentations. Batch, fed-batch and other approaches to fermentation practices are common and well known in the art and examples may be found in Thomas D. Brock in Biotechnology: A Textbook of Industrial Microbiology, Second Edition (1989) Sinauer Associates, Inc., Sunderland, Mass., Deshpande, Mukund V., Appl. Biochem. Biotechnol., 36:227, (1992), and Biochemical Engineering Fundamentals, 2nd Ed. J. E. Bailey and D. F. Ollis, McGraw Hill, New York, 1986, herein incorporated by reference for general instruction on biosynthesis of chemical products.

In accordance with the fermentation process of the current invention, the cells produce 3-HP during fermentation. Because of its acidic nature, as the concentration of 3-HP builds in the fermentation broth, the pH will decrease. Traditionally, a titrant such as ammonium hydroxide is added to the fermentation broth to maintain a desired pH. Applicants have discovered that the use of certain chemicals as titrants will enhance the production of 3-HP during the fermentation process.

Applicants have discovered that the use of certain carbonates (either alone or in combination with other bases) as a titrant during fermentation will enhance the production of 3-HP. Carbonates useful in the present invention preferably have a pH of greater than or equal to about 9.5, and more preferably greater than or equal to about 10, and have a water solubility of greater than or equal to about 1 mole/L at 30° C. In addition, carbonates that contain sodium, magnesium or calcium are particularly useful in the present invention. Preferably, the carbonate should not contain any potassium and should not be used in combination with other potassium containing bases. Chemicals that are particularly useful in the present invention include any sodium carbonate (e.g., sodium carbonate [Na₂CO₃], sodium bicarbonate [NaHCO₃], and sodium sesquicarbonate [Na₃H(CO₃)₂]), magnesium carbonate (e.g., magnesium carbonate [MgCO₃] and magnesium bicarbonate [Mg(HCO₃)₂]), or calcium carbonate (e.g., calcium carbonate [CaCO₃] and calcium bicarbonate [Ca(HCO₃)₂]), used either alone or in combination with one another or in combination with another base. When titrants in accordance with the present invention are used to maintain the pH during a 3-HP fermentation process, the amount of 3-HP produced will be increased by about 25% to 50% as compared to the same process utilized a traditional titrant such as ammonia hydroxide.

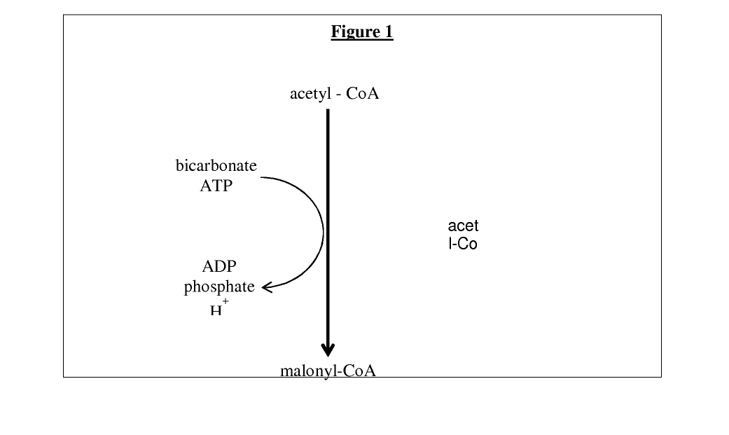

One of the key steps in the conversion of biomass to 3-HP is the conversion of acetyl-CoA to malonyl-CoA, which is illustrated in FIG. 1.

As shown, this reaction is catalyzed by the acetyl-CoA carboxylase, and bicarbonate is a reactant needed to drive the reaction. The use of a carbonate titrant can provide additional bicarbonate to facilitate the conversion of acetyl-CoA to malonyl-CoA. For example, when sodium carbonate (Na2CO3) is used as the fermentation titrant, and neutral pH is maintained, the sodium carbonate is converted to bicarbonate, creating an increased concentration of sodium bicarbonate in the fermentation media.

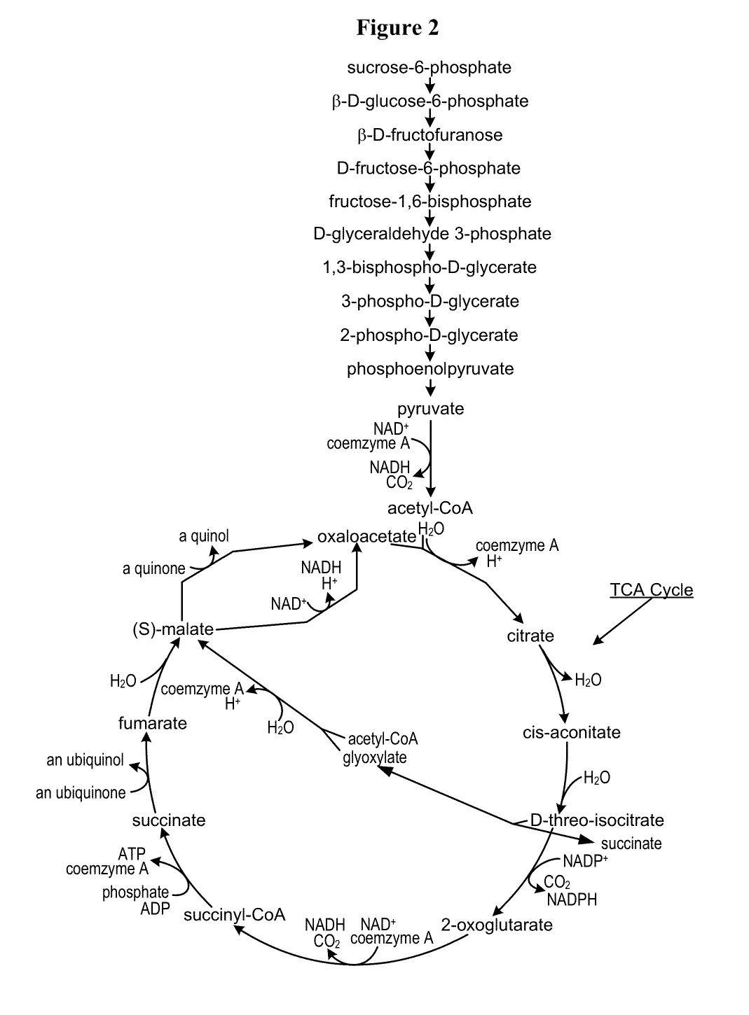

By contrast, in a traditional ammonium hydroxide system, bicarbonate for the acetyl-CoA→malonyl-CoA reaction is obtained through the natural metabolism of glucose to create carbon dioxide, which then forms carbonic acid and bicarbonate in solution. A part of the glucose metabolism pathway is shown in FIG. 2. As shown, carbon dioxide is created in the conversion of pyruvate to acetyl-CoA and in several steps in the tricarboxylic acid cycle (TCA cycle) (e.g., the conversion of D-threo-isocitrate to 2-oxoglutarate, and the conversion of 2-oxoglutarate to to succinyl-CoA.) Since the TCA cycle consumes acetyl-CoA that could otherwise be converted to 3-HP, in accordance with certain embodiments of the present invention, the initial bacterial strain is genetically modified to minimize, and preferably eliminate, the carbon flux through the TCA cycle during a second phase in the product process in order to maximize carbon flux to the production of 3-HP. In so doing, carbon dioxide from the TCA cycle is no longer available for conversion to bicarbonate for use in the acetyl-CoA to malonyl-CoA reaction. Use of the carbonate titrant in accordance with the present invention is particularly useful in combination with such a genetically modified organism that inhibits the TCA, since the carbonate titrant can provide additional bicarbonate to compensate for the carbon dioxide that would have been generated through the TCA cycle.

In accordance with the present invention, a carbonate titrant is used in conjunction with a genetically modified organism that down regulates during at least part of the process any one or more of the enzymes used in the TCA cycle, including citrate synthase (gltA), citrate hydro-lyase (acnA, acnB), isocitrate lyase (aceA), isocitrate dehydrogenase (icd), 2-oxoglutarate dehydrogenase (lpd), succinyl-CoA synthetase (sucD, sucC), succinate dehydrogenase (sdhA, sdhB, sdhC, sdhD), fumarase (fumA, fumB, fumC), malate synthase (aceB), and malate dehydrogenase (mdh). Preferably, the carbonate titrant is used in combination with a genetically modified organism that down regulates an enzyme that leads to the product of carbon dioxide, including citrate synthase (gltA), citrate hydro-lyase (acnA, acnB), isocitrate lyase (aceA), isocitrate dehydrogenase (icd), 2-oxoglutarate dehydrogenase (lpd).

The applicants have also discovered that carbonate, and in particular sodium carbonate, enhances the redox potential of NADH and NADPH. The final steps of the production of 3-HP during the fermentation process is shown below:

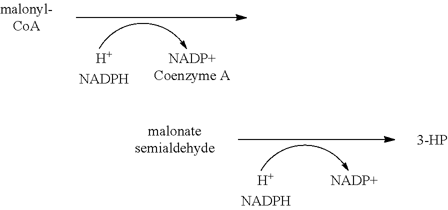

The enhanced redox potential of NADH and NADPH results in an increase in the 3-HP production.

In accordance with another aspect of the present invention, a microorganism is provided that includes a heterologous gene encoded therein that acts as a carbon dioxide importer (i.e., it enhances the importation of carbon dioxide into the cell or inhibits the exportation of carbon dioxide from the cell), which results in increased intracellular carbon dioxide. Carbon dioxide is readily diffusible through a cell's membrane, and a natural equilibrium will be reached between the intracellular and extracellular carbon dioxide. As a cell produces carbon dioxide it migrates through the cell, and since it is not very soluble in the media, it will bubble out of the system and more intracellular carbon dioxide with migrate out of the cell to maintain the equilibrium. This process impedes the production of 3-HP since bicarbonate (which is in equilibrium with the dissolved CO2 in the form of carbonic acid) is needed to drive the acetyl-CoA malonyl-CoA reaction and the intracellular carbon dioxide is the primary source for intracellular bicarbonate. Use of an importer gene mitigates against the natural outflow of carbon dioxide. In accordance with a preferred embodiment of the present invention, a microorganism is genetically modified to encode one or more of the following heterologous genes: bicA from Synechococcus species, ychM gene product of E. coli, yidE gene product of E. coli, or other examples of bicarbonate transporters as described in [Felce and Saier, J. Mol. Microbiol. Biotechnol. 8: 169-176, 2004], all of which will function as a carbon dioxide importer.

The following published resources are incorporated by reference herein for their respective teachings to indicate the level of skill in these relevant arts, and as needed to support a disclosure that teaches how to make and use methods of industrial biosynthesis of 3-HP, or other product(s) produced under the invention, from sugar sources, and also industrial systems that may be used to achieve such conversion with any of the recombinant microorganisms of the present invention (Biochemical Engineering Fundamentals, 2nd Ed. J. E. Bailey and D. F. Ollis, McGraw Hill, New York, 1986, entire book for purposes indicated and Chapter 9, pages 533-657 in particular for biological reactor design; Unit Operations of Chemical Engineering, 5th Ed., W. L. McCabe et al., McGraw Hill, New York 1993, entire book for purposes indicated, and particularly for process and separation technologies analyses; Equilibrium Staged Separations, P. C. Wankat, Prentice Hall, Englewood Cliffs, N.J. USA, 1988, entire book for separation technologies teachings). Generally, it is further appreciated, in view of the disclosure, that any of the above methods and systems may be used for production of chemical products other than 3-HP that require bicarbonate or carbon dioxide or require enhanced redox potential of NADH or NADPH.

It is noted that embodiments of the invention may be practiced in large-scale fermentation vessels, such as steel vessels, for cost-effective commercial production of a selected chemical product. For example, a steel or other vessel may be greater than 250 L, greater than 1,000 L, greater than 10,000 L, greater than 50,000 L, greater than 100,000 L or greater than 200,000 L. The specific examples below are not intended to limit the scope of size of vessels in which the any embodiment of the invention may be practiced.

### Bio-Production Reactors and Systems

Fermentation systems utilizing methods and/or compositions according to the invention are also within the scope of the invention.

Any of the recombinant microorganisms as described and/or referred to herein may be introduced into an industrial bio-production system where the microorganisms convert a carbon source into a selected chemical product, such as 3-HP, in a commercially viable operation. The bio-production system includes the introduction of such a recombinant microorganism into a bioreactor vessel, with a carbon source substrate and bio-production media suitable for growing the recombinant microorganism, and maintaining the bio-production system within a suitable temperature range (and dissolved oxygen concentration range if the reaction is aerobic or microaerobic) for a suitable time to obtain a desired conversion of a portion of the substrate molecules to 3-HP. Industrial bioproduction systems and their operation are well-known to those skilled in the arts of chemical engineering and bioprocess engineering.

Bio-productions may be performed under aerobic, microaerobic, or anaerobic conditions, with or without agitation. The operation of cultures and populations of microorganisms to achieve aerobic, microaerobic and anaerobic conditions are known in the art, and dissolved oxygen levels of a liquid culture comprising a nutrient media and such microorganism populations may be monitored to maintain or confirm a desired aerobic, microaerobic or anaerobic condition. When syngas is used as a feedstock, aerobic, microaerobic, or anaerobic conditions may be utilized. When sugars are used anaerobic, aerobic or microaerobic conditions can be implemented in various embodiments. Any of the recombinant microorganisms as described and/or referred to herein may be introduced into an industrial bio-production system where the microorganisms convert a carbon source into 3-HP, and optionally in various embodiments also to one or more downstream compounds of 3-HP in a commercially viable operation.

In various embodiments, syngas components or sugars are provided to a microorganism, such as in an industrial system comprising a reactor vessel in which a defined media (such as a minimal salts media including but not limited to M9 minimal media, potassium sulfate minimal media, yeast synthetic minimal media and many others or variations of these), an inoculum of a microorganism providing an embodiment of the biosynthetic pathway(s) taught herein, and the carbon source may be combined. The carbon source enters the cell and is catabolized by well-known and common metabolic pathways to yield common metabolic intermediates, including phosphoenolpyruvate (PEP). See Molecular Biology of the Cell, 3rd Ed., B. Alberts et al. Garland Publishing, New York, 1994, pp. 42-45, 66-74, incorporated by reference for the teachings of basic metabolic catabolic pathways for sugars; Principles of Biochemistry, 3rd Ed., D. L. Nelson & M. M. Cox, Worth Publishers, New York, 2000, pp 527-658, incorporated by reference for the teachings of major metabolic pathways; and Biochemistry, 4th Ed., L. Stryer, W. H. Freeman and Co., New York, 1995, pp. 463-650, also incorporated by reference for the teachings of major metabolic pathways.).)

Further to types of industrial bio-production, various embodiments of the present invention may employ a batch type of industrial bioreactor. A classical batch bioreactor system is considered ‘“closed” meaning that the composition of the medium is established at the beginning of a respective bio-production event and not subject to artificial alterations and additions during the time period ending substantially with the end of the bio-production event. Thus, at the beginning of the bio-production event the medium is inoculated with the desired organism or organisms, and bio-production is permitted to occur without adding anything to the system. Typically, however, a “batch” type of bio-production event is batch with respect to the addition of carbon source and attempts are often made at controlling factors such as pH and oxygen concentration. In batch systems the metabolite and biomass compositions of the system change constantly up to the time the bio-production event is stopped. Within batch cultures cells moderate through a static lag phase to a high growth log phase and finally to a stationary phase where growth rate is diminished or halted. If untreated, cells in the stationary phase will eventually die. Cells in log phase generally are responsible for the bulk of production of a desired end product or intermediate. Alternatively, however, cells in stationary phase maybe responsible for the bulk of production. In accordance with this invention, bioproduction may be either growth associated production (i.e., simultaneous replication of cells and production of the chemical product of interest) or non-growth associated production (i.e., production of the chemical product of interest after the replication of the cells substantially ceases).

A variation on the standard batch system is the fed-batch system. Fed-batch bio-production processes are also suitable in the present invention and comprise a typical batch system with the exception that some or all of the nutrients, including the substrate, are added in increments as the bio-production progresses. Fed-Batch systems are useful when catabolite repression is apt to inhibit the growth and/or metabolism of the cells and where it is desirable to have limited amounts of substrate in the media. Measurement of the actual nutrient concentration in Fed-Batch systems may be measured directly, such as by sample analysis at different times, or estimated on the basis of the changes of measurable factors such as pH, dissolved oxygen and the partial pressure of waste gases such as carbon dioxide. Batch and fed-batch approaches are common and well known in the art and examples may be found in Thomas D. Brock in Biotechnology: A Textbook of Industrial Microbiology, Second Edition (1989) Sinauer Associates, Inc., Sunderland, Mass., Deshpande, Mukund V., Appl. Biochem. Biotechnol., 36:227, (1992), and Biochemical Engineering Fundamentals, 2nd Ed. J. E. Bailey and D. F. Ollis, McGraw Hill, New York, 1986, herein incorporated by reference for general instruction on bio-production.

Although embodiments of the present invention may be performed in batch mode, or in fed-batch mode, it is contemplated that the invention would also be adaptable to continuous bio-production methods. Continuous bioproduction is considered an “open” system where a defined bio-production medium is added continuously to a bioreactor and an equal amount of conditioned media is removed simultaneously for processing. Continuous bioproduction generally maintains the cultures within a controlled density range where cells are primarily in log phase growth. There are two types of continuous bioreactor operations—chemostat and perfusion culture. In a chemostat operation fresh media is fed to the vessel while simultaneously removing an equal rate of the vessel contents. The limitation of this approach is that cells are lost and high cell density generally is not achievable. In fact, typically one can obtain much higher cell density with a fed-batch process. A perfusion culture operation is similar to the chemostat approach except that the stream that is removed from the vessel is subjected to a separation technique which recycles viable cells back to the vessel. This type of continuous bioreactor operation has been shown to yield significantly higher cell densities than fed-batch and can be operated continuously. Continuous bioproduction is particularly advantageous for industrial operations because it has less down time associated with draining, cleaning and preparing the equipment for the next bio-production event. In addition, the biocatalyst does not have to be regenerated, saving time and cost associated with biomass growth. Furthermore, it is typically more economical to continuously operate downstream unit operations, such as distillation, than to run them in batch mode.

Continuous bio-production allows for the modulation of one factor or any number of factors that affect cell growth or end product concentration. For example, one method will maintain a limiting nutrient such as the carbon source or nitrogen level at a fixed rate and allow all other parameters to moderate. In other systems a number of factors affecting growth can be altered continuously while the cell concentration, measured by media turbidity or some other equivalent method, is kept constant. Methods of modulating nutrients and growth factors for continuous bio-production processes as well as general techniques for maximizing the rate of product formation are known in the art of industrial microbiology and a variety of such methods are detailed by Brock, supra.

It is contemplated that embodiments of the present invention may be practiced using either batch, fed-batch or continuous processes and that any known mode of bio-production would be suitable. It is contemplated that cells may be immobilized on an inert scaffold as whole cell catalysts and subjected to suitable bio-production conditions for 3-HP production, or be cultured in liquid media in a vessel, such as a culture vessel. Thus, embodiments used in such processes, and in bio-production systems using these processes, include a population of genetically modified microorganisms of the present invention, a culture system comprising such population in a media comprising nutrients for the population, and methods of making 3-HP and thereafter, a downstream product of 3-HP. Embodiments of the invention include methods of making 3-HP in a bio-production system, some of which methods may include obtaining 3-HP after such bio-production event. For example, a method of making 3-HP may comprise: providing to a culture vessel a media comprising suitable nutrients; providing to the culture vessel an inoculum of a genetically modified microorganism comprising genetic modifications described herein such that the microorganism produces 3-HP from syngas and/or a sugar molecule; and maintaining the culture vessel under suitable conditions for the genetically modified microorganism to produce 3-HP.

It is within the scope of the present invention to produce, and to utilize in bio-production methods and systems, including industrial bio-production systems for production of 3-HP, a recombinant microorganism genetically engineered to modify one or more aspects effective to increase production of 3-HP by at least 25 percent over control microorganism lacking the one or more modifications. In various embodiments, the invention is directed to a system for bioproduction of acrylic acid as described herein, said system comprising: a fermentation tank suitable for microorganism cell culture; a line for discharging contents from the fermentation tank to an extraction and/or separation vessel; an extraction and/or separation vessel suitable for removal of 3-hydroxypropionic acid from cell culture waste; a line for transferring 3-hydroxypropionic acid to a dehydration vessel; and a dehydration vessel suitable for conversion of 3-hydroxypropionic acid to acrylic acid. In various embodiments, the system includes one or more pre-fermentation tanks, distillation columns, centrifuge vessels, back extraction columns, mixing vessels, or combinations thereof.

Also, it is within the scope of the present invention to produce, and to utilize in bio-production methods and systems, including industrial bio-production systems for production of a selected chemical product other than 3-HP, a recombinant microorganism genetically engineered to modify one or more aspects effective to increase the selected chemical product's bio-production by at least 25 percent over control microorganism lacking the one or more modifications.

In various embodiments, the invention is directed to a system for bio-production of a chemical product as described herein, said system comprising: a fermentation tank suitable for microorganism cell culture; a line for discharging contents from the fermentation tank to an extraction and/or separation vessel; and an extraction and/or separation vessel suitable for removal of the chemical product from cell culture waste. In various embodiments, the system includes one or more pre-fermentation tanks, distillation columns, centrifuge vessels, back extraction columns, mixing vessels, or combinations thereof.

The following published resources are incorporated by reference herein for their respective teachings to indicate the level of skill in these relevant arts, and as needed to support a disclosure that teaches how to make and use methods of industrial bio-production of 3-HP, or other product(s) produced under the invention, from sugar sources, and also industrial systems that may be used to achieve such conversion with any of the recombinant microorganisms of the present invention (Biochemical Engineering Fundamentals, 2nd Ed. J. E. Bailey and D. F. Ollis, McGraw Hill, New York, 1986, entire book for purposes indicated and Chapter 9, pages 533-657 in particular for biological reactor design; Unit Operations of Chemical Engineering, 5th Ed., W. L. McCabe et al., McGraw Hill, New York 1993, entire book for purposes indicated, and particularly for process and separation technologies analyses; Equilibrium Staged Separations, P. C. Wankat, Prentice Hall, Englewood Cliffs, N.J. USA, 1988, entire book for separation technologies teachings). Generally, it is further appreciated, in view of the disclosure, that any of the above methods and systems may be used for production of chemical products other than 3-HP.

### EXAMPLES

### General Experimental Protocol

Examples 1-5 relate to experiments conducted using batch Applikon or Das Gip fermentors. The following general procedures apply to these examples.

The Applikon 3 L (nominal volume) fermentors are typically run with a 1.6 L initial batch volume, while the Das-Gip 1 L (nominal volume) fermentors are typically run with a 0.7 L initial batch volume. In each case broth is removed as required during the fed-batch portion of the fermentation to maintain the working volume below the fermentor's maximum capacity.

Cultures are grown in the following (FM7) medium: 3 g/L ammonium sulfate, 1.8 g/L citric acid (anhydrous), 0.8 g/L magnesium sulfate heptahydrate, 5 mL/L trace metals solution, 1.25 mL/L vitamin solution, 0.8 mL/L antibiotics and the desired amounts of glucose and potassium phosphate monobasic. The vitamin solution contains 2 g/L thiamine hydrochloride, 0.5 g/L D-pantothenic acid, 0.5 g/L nicotinic acid and 0.2 g/L biotin. The trace metals solution contains 19.2 g/L citric acid (anhydrous), 2.0 g/L ferrous sulfate heptahydrate, 0.086 g/L zinc sulfate heptahydrate, 0.062 g/L manganese sulfate monohydrate, 0.10 g/L cupric sulfate pentahydrate, and 6.67 g/L calcium carbonate. Antibiotics are 35 mg/mL kanamycin and 20 mg/mL chloramphenicol. The initial concentration of glucose is typically 30 g/L. Throughout the fermentation the cultures are fed a 60% (w/w) glucose solution to maintain a positive residual glucose concentration, typically 5-20 g/L.

Cultures are grown at 30° C. until the initial batched phosphate has depleted, and then the cultures are switched to 37° C. for the duration of the production phase. The amount of phosphate in the batch medium typically ranges from 9-18 mM, and the growth phase typically lasts between 12-20 h, though this is highly strain dependent. Unless otherwise noted, during growth the pH is controlled using a 75:25 mixture of concentrated ammonium hydroxide and water.

Dissolved oxygen is maintained at the desired levels (between 20-50% during growth) by controlling agitation and sparging with sterile-filtered air, supplemented with oxygen if needed. During production a microaerobic environment is maintained either by controlling the dissolved oxygen at low levels (1-5%) or by controlling the ORP (oxidative-reductive potential) of the culture at circa −200 mV.

### Example 1

### Fermentation Study Using Various Titrants

### Based on Experiment Number: 20121217-AN

A study was conducted to determine the effect of various base titrants on the production of 3-HP. The general experimental protocol noted above was used in this experiment with 1.6 L fermentors. The genetically modified E. coli strain used had the following genetic modifications:

| Genotype | Plasmids |
|---|---|
| F-, Δ(araD-araB)567, ΔlacZ4787(::rrnB-3), LAM-, | (1) pTRCΔlacI-kan-PyibD-(Ca)Mcr |
| rph-1, Δ(rhaD-rhaB)568, hsdR514, ΔldhA::frt, | (2) pACYC-cam-Ptal:pntAB/ |
| ΔpflB::frt, ΔmgsA::frt, ΔpoxB::frt, Δpta-ack::frt, | PtpiA:accAD_PrpiA:accBC |
| fabI(ts)-(S241F)-zeoR, fabB(ts), ΔfabF::frt, coaA*, | |
| fabD(ts) | |

*mutation- residue R106-A

During the production phase the following base titrants where used:

| Sample# | Base Titrant - Production Phase |
|---|---|
| 1 | 75% (6M) NH₄OH, +2M NaHCO₃ FEED |
| 1A | 75% (6M) NH₄OH, +2M NaHCO₃ FEED |
| 2 | 75% (6M) NH₄OH |
| 3 | 2M Na₂CO₃ |
| 4 | 2M Na₂CO₃, +2M NaHCO₃ FEED |
| 5 | 6M NaOH, +2M NaHCO₃ FEED |
| 5A | 6M NaOH, +2M NaHCO₃ FEED |
| 7 | 6M NaOH |
| 8 | 6M K₂CO₃ |
| 9 | 6M (NH₄)₂CO₃ |

In the samples were sodium bicarbonate was feed, there was a continuous feed of the bicarbonate at a rate of 1 mole per 24 hours beginning at induction with NH₄OH, NaOH or Na₂CO₃ as base titrants.

The results of the study are show in the following table:

| | | | Max | | Total | |
|---|---|---|---|---|---|---|
| | | | 3HP | Volume | grams | Relative |
| Sample | Titrant | Feed | titer | increase | 3HP | % |
| 1 | NH₄OH | NaHCO₃ | 53.0 | 75% | 148.5 | 159% |
| 1A | NH₄OH | NaHCO₃ | 41.7 | 74% | 116.4 | 125% |
| 2 | NH₄OH | na | 47.1 | 24% | 93.4 | 100% |
| 3 | Na₂CO₃ | na | 62.0 | 50% | 148.7 | 159% |
| 4 | Na₂CO₃ | NaHCO₃ | 47.1 | 86% | 140.0 | 150% |
| 5 | NaOH | NaHCO₃ | 43.9 | 77% | 124.6 | 133% |
| 5A | NaOH | NaHCO₃ | 45.6 | 85% | 135.0 | 145% |
| 6 | NaOH | na | 36.2 | 41% | 81.7 | 88% |
| 7 | K₂CO₃ | na | 33.2 | 25% | 66.4 | 71% |
| 8 | (NH₄)₂CO₃ | na | 44.5 | 27% | 90.3 | 97% |

Of the titrants included in this study, sodium carbonate (sample 3) had as significant impact on the 3-HP production as compared to the other titrants. Samples 1, 1A, 5, and 5A tested the impact of feeding NaHCO₃ in addition to the ammonium hydroxide and sodium hydroxide base titrants. In each sample the addition of NaHCO₃ had a significant positive effect on the total amount of 3HP produced, resulting in 25-59% increase in total 3HP accumulated. In contrast, however, in sample 4 the addition of NaHCO₃ did not have a significant effect on the total amount of 3HP produced when using Na₂CO₃ as the production phase base titrant. The volume increase observed for the Na₂CO₃ tank fed NaHCO₃ (sample 4) was 86% vs. 50% for its no feed control (sample 3).

The tank utilizing K₂CO₃ as the production phase titrant (sample 7) significantly underperformed all of the other tanks in terms of titer, total 3HP produced and production phase yield. The presence of potassium in the culture is believed to be inhibitory of the 3-HP production pathway.

### Example 2

### Comparison of Sodium Carbonate and Ammonium Hydroxide

### Based on Experiment Number: 20130121-AN

A study was conducted to determine the effect of using sodium carbonate versus ammonium hydroxide as the base titrant during the production phase of the fermentation to produce 3-HP. The general experimental protocol noted above was used in this experiment. The genetically modified E. coli strain used had the following genetic modifications:

| Genotype | Plasmids |
|---|---|
| F-, Δ(araD-araB)567, | 1. pTRCΔlacI-kan-PyibD-(St)Mcr_PyibD- |
| ΔlacZ4787(::rrnB-3), | NDSD |
| LAM-, rph-1, | 2. pJ251-cam-PtpiA:accAD_PrpiA:accBC |
| Δ(rhaD-rhaB)568, | |
| hsdR514, ΔldhA::frt, | |
| ΔpflB::frt, ΔmgsA::frt, | |
| ΔpoxB::frt, Δpta-ack::frt, | |
| fabI(ts)-(S241F)-zeoR | |

The following samples were included in this study:

| Sample # | Base during growth | Base during production |
|---|---|---|
| A10 | NH4OH | NH4OH |
| A3 | NH4OH | Na2CO3 |
| A7 | NH4OH | NH4OH |

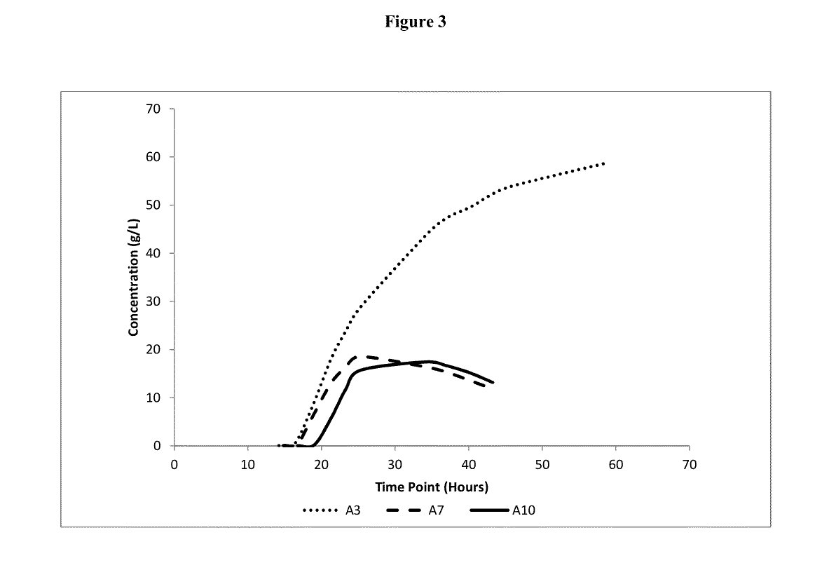

The results of this study are shown in FIG. 3.

As shown in FIG. 3, the sample that used the Na₂CO₃ as the titrant (sample A3) produced significantly more 3-HP, and did not show a cessation of production. The samples using NH₄OH as the titrant (samples A7 and A10) proved to not only negatively affect production but also showed that cells were no longer metabolically active through a rise in pH and cessation of glucose consumption.

### Example 3

### Comparison of Various Base Titrants

### Based on Experiment Number: 20130121-AN

A study was conducted to determine the effect of using various base titrants during the production phase of the fermentation to produce 3-HP. The general experimental protocol noted above was used in this experiment. The genetically modified E. coli strain used had the following genetic modifications:

| Genotype | Plasmids |
|---|---|
| F-, Δ(araD-araB)567, ΔlacZ4787(::rrnB-3), | 1. pET28ΔlacI-PpstsIH- |
| LAM-, rph-1, Δ(rhaD-rhaB)568, hsdR514, | (St)Mcr-rrnbTT_T5- |
| ΔldhA::frt, ΔpflB::frt, ΔmgsA::frt, ΔpoxB::frt, | ydfG |
| Δpta-ack::frt, fabI(ts)-(S241F)-zeoR, ΔyieP::frt | 2. pACYC-cam- |
| | Ptal:pntAB/ |
| | PtpiA:accAD- |
| | PrpiA:accBC |

The following samples were included in this study:

| Sample | Base during production |
|---|---|
| 1 | NH₄OH - control |
| 2 | NaHCO₃ |
| 3 | NaHCO₃ |
| 4 | NH₄OH + 3M (NH₄)HCO₃ |
| 5 | NH₄OH + 1M (NH₄)HCO₃ |

For samples A8 and A9, the ammonium bicarbonate was spiked into the reaction vessel at 1 hour after the temperature shift that initiated the production phase.

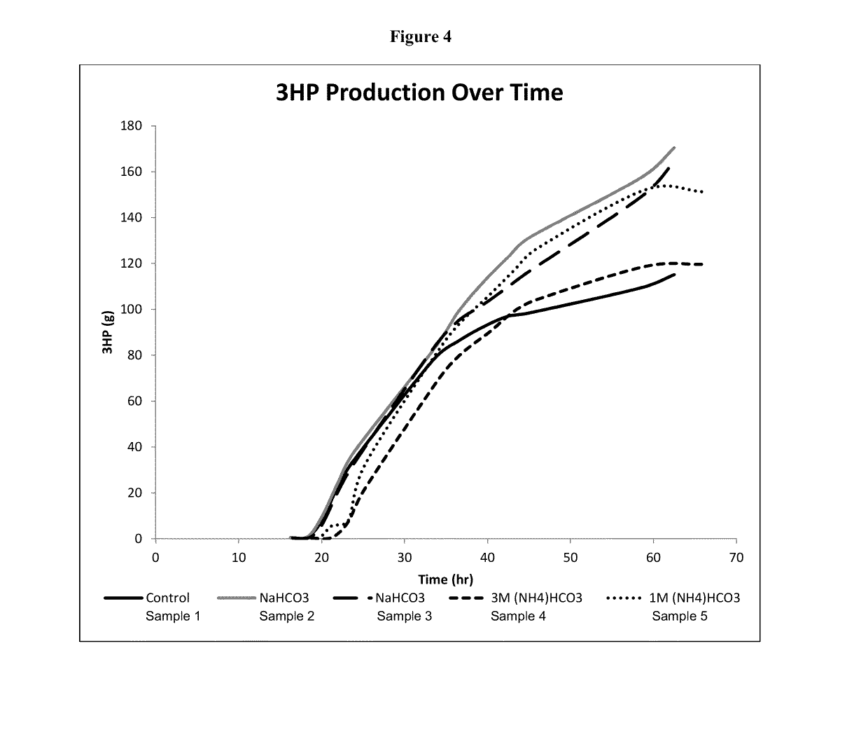

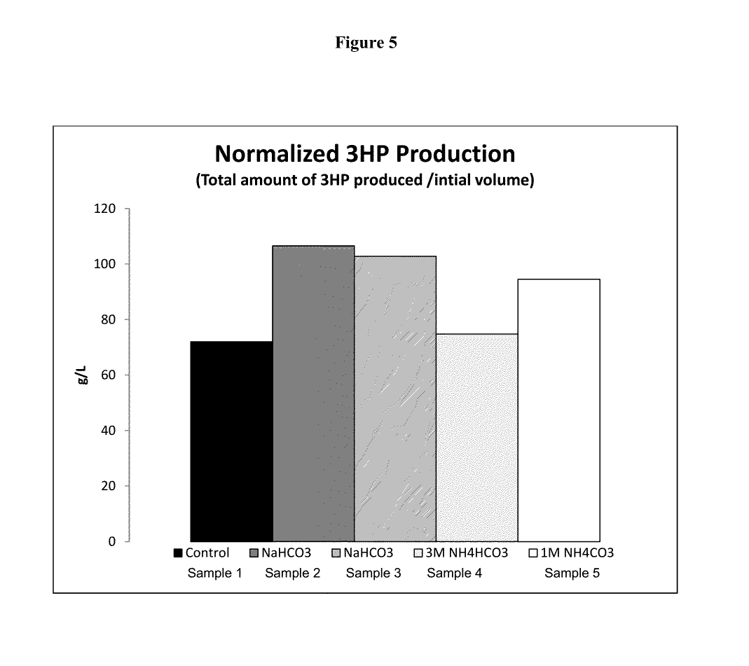

The results of this study are show in FIGS. 4-5.

### Example 5

### Evaluation of the Impact of Excess Ammonium

### Based on Experiment Number: 201300204-AN

A study was conducted to determine the effect of using excess ammonium in the base titrant during the production phase of the fermentation to produce 3-HP. The general experimental protocol noted above was used in this experiment with 1.6 L fermentors. The genetically modified E. coli strain used had the following genetic modifications:

| Genotype | Plasmids |
|---|---|
| F-, Δ(araD-araB)567, | (1) pTRCΔlacI-kan- |
| ΔlacZ4787(::rrnB-3), LAM-, rph-1, | PyibD-(Ca)Mcr |
| Δ(rhaD-rhaB)568, hsdR514, | (2) pACYC-cam- |
| ΔldhA::frt, ΔpflB::frt, ΔmgsA::frt, | Ptal:pntAB/PtpiA:acc |
| ΔpoxB::frt, Δpta-ack::frt, fabI(ts)- | AD-PrpiA:accBC |
| (S241F)-zeoR | |

Fermentation runs were conducted using the following samples:

| | Base during | |
|---|---|---|
| Sample # | production | Additional Titrant |
| A1 | NH₄OH | none |
| A3 | NH₄OH | none |
| A4 | Na₂CO₃ | none |
| A5 | Na₂CO₃ | None |
| A6 | NH₄OH | 1L of 1M NH₄HCO₃ at 3 rpm for 20 h, starting |
| | | when redox is under control |
| A7 | NH₄OH | 1L of 1M NH₄HCO₃ at 3 rpm for 20 h, starting |
| | | when redox is under control |
| A8 | NH₄OH | 1L of 1M NaHCO₃ at 3 rpm for 20 h, starting |
| | | when redox is under control |
| A9 | NH₄OH | 1L of 1M NaHCO₃ at 3 rpm for 20 h, starting |
| | | when redox is under control |

The amount of ammonium present in each run at 37 hours was determined and is show in the following table:

| | | Mole | Mole | | | |
|---|---|---|---|---|---|---|
| | NH₄ ml | added | NH₄ | | | |
| | added | through | added | Total | Volume | |
| Sample | at 37 hrs | base | by feed | NH₄ | at 37 hrs | [NH₄] M |
| a1 | 159. | 0.957 | 0 | 0.957 | 2.09 | 0.45730659 |
| a3 | 11 | 0.70 | 0 | 0.70 | 2.01 | 0.351364764 |
| a6 | 83. | 0.498 | 1 | 1.498 | 3.02 | 0.495077635 |
| a7 | 76.4 | 0.458 | 1 | 1.458 | 3.06 | 0.476134509 |
| a4 | 34.0 | 0.20 | | 0.20 | 2.48 | 0.082059533 |
| a5 | 32.0 | 0.19 | | 0.19 | 2.4 | 0.07804878 |
| a8 | 85.4 | 0.512 | | 0.512 | 3.03 | 0.168663594 |
| a9 | 80.0 | 0.4 | | 0.4 | 3.03 | 0.158206987 |

The runs were then grouped into three categories based on ammonium levels: high (A1, A3, A6, and A7), low (A8 and A9), and trace (A4 and A5).

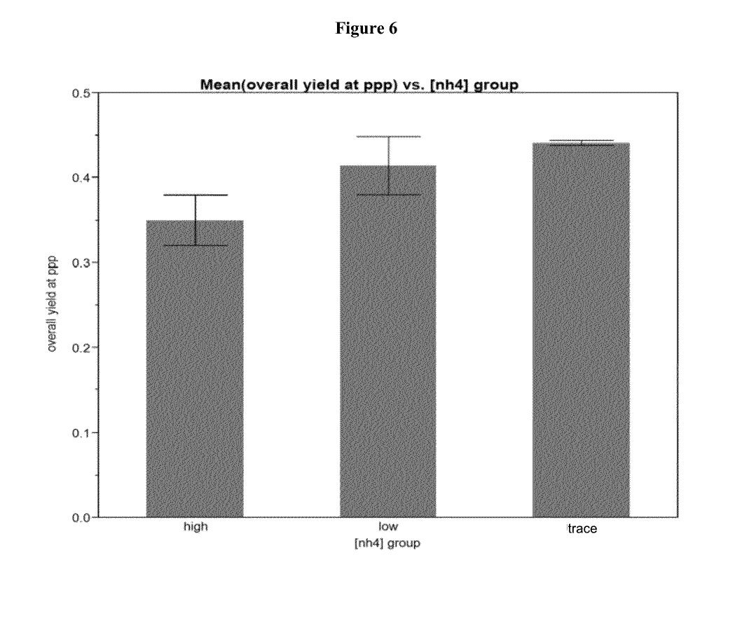

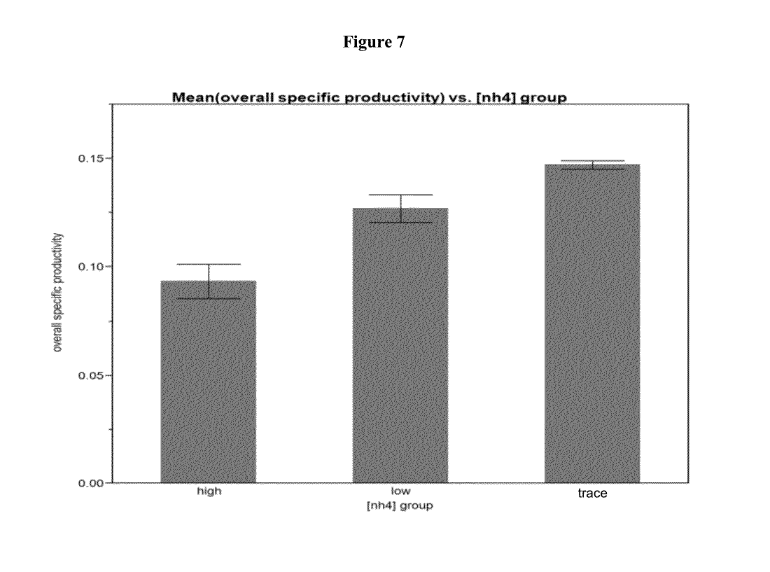

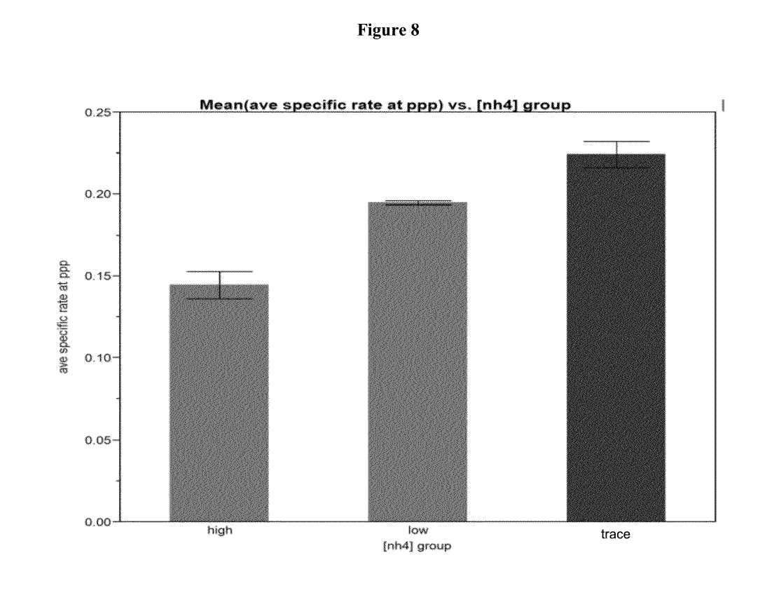

The results of this are shown in FIGS. 6-8. The results clearly show that increased ammonium adversely affects 3-HP production. Therefore, although ammonium carbonate may be used as an effective titrant, in accordance with a preferred embodiment the concentration of the ammonium ion in the broth should be less than about 0.2 M, or about 0.15M, or about 0.1.

### Example 6

### Shake Flasks Study Using Various Concentrations of Sodium Carbonate

### Based on 20120524_SDTeam_036

This experiment sought to determine whether the addition of Na₂CO₃ to a 3-HP shake flask fermentation would result in higher 3-HP titers and prolonged 3-HP production. The following strain was tested:

| Genotype | Plasmid(s) |
|---|---|
| F-, Δ(araD-araB)567, ΔlacZ4787(::rrnB-3), | pTRC-KAN-ptrc-mcr, |
| LAM-, rph-1, Δ(rhaD-rhaB)568, hsdR514, | pACYC-CAT- |
| ΔldhA::frt, ΔpflB::frt, ΔmgsA::frt, ΔpoxB::frt, | Ptal:pntAB/ |
| Δpta-ack::frt, fabI(ts)-(S241F)-zeoR | PtpiA:accAD- |
| | PrpiA: accBC |

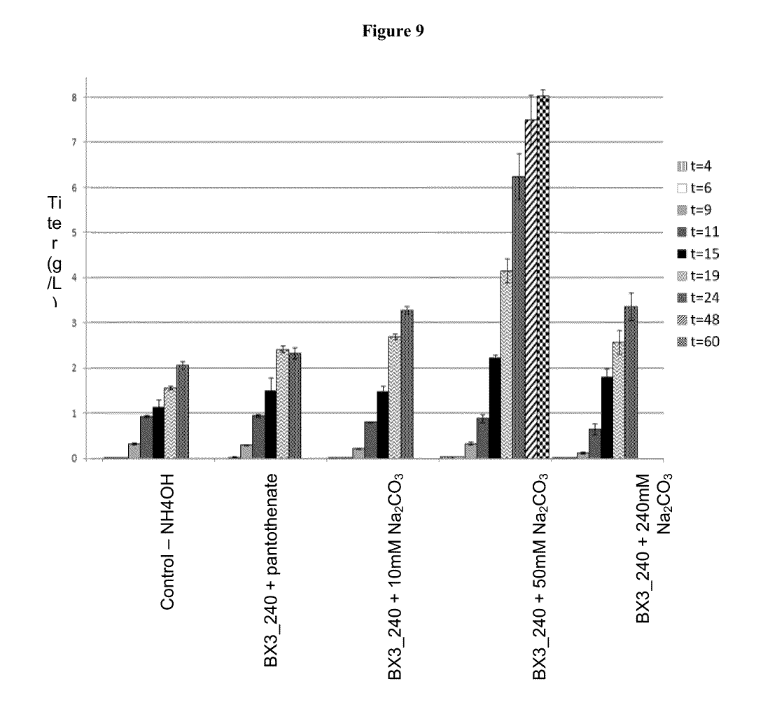

The above strain was evaluated in shake flasks for the production of 3-HP with various amounts of sodium carbonate added to the shake flasks (10 mM, 20 mM, and 50 mM Na₂CO₃). Triplicate evaluations were performed. Overnight starter cultures were made in 50 mL of Luria Broth including the appropriate antibiotics and incubated 16-24 hours are 30° C., while shaking at 225 rpm. These cultures were used to inoculate 3×50 mL cultures of each strain in medium with 5% culture as the starting inoculum, antibiotics, and 1 mM IPTG. Flasks were grown at 30° C. in a shaking incubator. At 4, 6, 9, 11, 15, 19, 25, 48 and 60 hours samples were taken for analyses of OD at 600 nm and 3-HP production using the 3-HP bioassay described in the Common Methods Section. The results are shown in FIG. 9.

The samples that included sodium carbonate had increased 3-HP titers, with the sample having 50 mM sodium carbonate showing a significant increase in 3-HP titers and prolonged 3-HP production.

While preferred embodiments of the present invention have been shown and described herein, any aspect of the current invention may be combined with one or more of any other aspect of the current invention. In addition, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of producing a fermentation product using an organism genetically modified for the conversion of acetyl-CoA to malonyl-CoA, the method comprising the steps of:
a) growing the organism in the presence of a carbon source and source of ammonium;
b) producing the fermentation product while culturing the organism in presence of a carbon source and a non-potassium carbonate titrant.

2. The method of claim 1, wherein the non-potassium carbonate titrant has a pH of at least 9.5.

3. The method of claim 2, wherein the non-potassium carbonate titrant comprises sodium, magnesium, calcium, or mixtures thereof.

4. The method of claim 1, wherein the non-potassium carbonate titrant has a water solubility of at least 1 mole/L at 30° C.

5. The method of claim 1, wherein the non-potassium carbonate titrant is sodium carbonate.

6. The method of claim 1, wherein the non-potassium carbonate titrant is selected from the group consisting of: sodium carbonate, sodium bicarbonate, sodium sesquicarbonate, magnesium carbonate, magnesium bicarbonate, calcium carbonate, and calcium bicarbonate.

7. The method of claim 1, wherein said method produces from 25% to 50% more fermentation product compared to the same process utilizing ammonia hydroxide as a titrant during step b).

8. The method of claim 1, wherein said organism is an organism selected from the group consisting of: a bacteria and a yeast.

9. The method of claim 1, wherein said organism is an organism selected from the group consisting of: E. coli, Cupriavidus necator, and Saccharomyces.

10. The method of claim 1, wherein said organism is genetically modified to down regulate one or more enzymes used in the TCA cycle.

11. The method of claim 10, wherein said one or more enzymes used in the TCA cycle are selected from the group consisting of: citrate synthase, citrate hydro-lyase, isocitrate lyase, isocitrate dehydrogenase, 2-oxoglutarate dehydrogenase, succinyl-CoA synthetase, succinate dehydrogenase, fumarase, malate synthase, and malate dehydrogenase.

12. The method of claim 1, wherein said organism is genetically modified to down regulate an enzyme that leads to the production of carbon dioxide.

13. The method of claim 12, wherein said enzyme that leads to the production of carbon dioxide is selected from the group consisting of: citrate synthase, citrate hydro-lyase, isocitrate lyase, isocitrate dehydrogenase, and 2-oxoglutarate dehydrogenase.

14. The method of claim 1, wherein said organism is genetically modified to include at least one nucleic acid encoding for polypeptide that functions as a carbon dioxide importer.

15. The method of claim 14, wherein said polypeptide that functions as a carbon dioxide importer increases intracellular carbon dioxide.

16. The method of claim 1, wherein said organism is genetically modified to include at least one nucleic acid selected from the group consisting of: bicA, ychM, and yidE.

17. The method of claim 1, wherein said titrant enhances the redox potential of NADH or NADPH.

18. The method of claim 1, wherein said culturing is performed under a condition selected from the group consisting of: aerobic, microaerobic, and anaerobic.

19. The method of claim 1, further comprising maintaining a dissolved oxygen concentration within 20-50%.

20. The method of claim 1, wherein said growth phase is conducted at a temperature from 25 to 30° C.

21. The method of claim 1, wherein the production phase is conducted at a temperature from 35 to 45° C.

22. The method of claim 1, wherein said production phase temperature is higher than said growth phase temperature.

23. The method of claim 1, wherein the organism comprises an exogenous polynucleotide encoding a polypeptide that is an acetyl-CoA carboxylase.
